(19) United States
(12) Patent Application Publication
(10) Pub. No.: US 2014/0011870 A1
(43) Pub. Date: Jan. 9, 2014

(54) Methods of Treating Obesity Using an Effective Dose of a METAP-2 Inhibitor

(71) Applicant: Thomas E. Hughes, Boston, MA (US)

(72) Inventor: Thomas E. Hughes, Boston, MA (US)

(73) Assignee: Zafgen, Inc., Cambridge, MA (US)

(21) Appl. No.: 13/990,277

(22) PCT Filed: Nov. 29, 2011
(86) PCT No.: PCT/US11/62421

§ 371 Date: Aug. 13, 2013

Related U.S. Application Data
(60) Provisional application No. 61/417,700, filed on Nov. 29, 2010.

(51) Int. Cl. A61K 31/336

(52) U.S. Cl. CPC A61K 31/336
USPC 514/475

(57) ABSTRACT

The invention generally relates to methods of treating an overweight or obese subject, and treating overweight- or obesity-related conditions using effective of amounts of a MetAP-2 inhibitor.

### CROSS REFERENCE TO RELATED APPLICATION

This application claims the benefit of and priority to U.S. Provisional Patent Application No. 61/417,700, filed Nov. 29, 2010, the content of which is hereby incorporated by reference in its entirety.

### BACKGROUND

Obesity is a complex medical disorder of appetite regulation and metabolism resulting in excessive accumulation of adipose tissue mass. Typically defined as a body mass index (BMI) of 30 kg/m² or more, obesity is a world-wide public health concern that is associated with cardiovascular disease, diabetes, certain cancers, respiratory complications, osteoarthritis, gallbladder disease, decreased life expectancy, and work disability. The primary goals of obesity therapy are to reduce excess body weight, improve or prevent obesity-related morbidity and mortality, and maintain long-term weight loss.

Treatment modalities typically include diet and exercise programs, lifestyle management, pharmacotherapy, and surgery. Treatment decisions are made based on severity of obesity, seriousness of associated medical conditions, patient risk status, and patient expectations. Notable improvements in cardiovascular risk and the incidence of diabetes have been observed with weight loss of 5-10% of body weight, supporting clinical guidelines for the treatment of obesity that recommend a target threshold of 10% reduction in body weight from baseline values. These improvements are notable even for patients who may be overweight (with a BMI of 27 kg/m²) but also have a weight related co-morbidity such as hypertension, type 2 diabetes, dyslipidemia, or central adiposity.

However, while prescription anti-obesity medications are typically considered for selected patients at increased medical risk because of their weight and for whom lifestyle modifications (diet restriction, physical activity, and behavior therapy) alone have failed to produce durable weight loss, approved drugs have had unsatisfactory efficacy for severely obese subjects, leading to only ˜3-5% reduction in body weight after a year of treatment.

Bariatric surgery may be considered as a weight loss intervention for patients at or exceeding a BMI of 40 kg/m². Patients with a BMI ≧35 kg/m² and an associated serious medical condition are also candidates for this treatment option. Unfortunately, postoperative complications commonly result from bariatric surgical procedures, including bleeding, embolism or thrombosis, wound complications, deep infections, pulmonary complications, and gastrointestinal obstruction; reoperation during the postoperative period is sometimes necessary to address these complications. Rates of reoperation or conversion surgery beyond the postoperative period depend on the type of bariatric procedure, and in one study ranged from 17% to 31%. Intestinal absorptive abnormalities, such as micronutrient deficiency and protein-calorie malnutrition, also are typically seen with bypass procedures, requiring lifelong nutrient supplementation. Major and serious adverse outcomes associated with bariatric surgery are common, observed in approximately 4 percent of procedures performed (including death in 0.3 to 2 percent of all patients receiving laparoscopic banding or bypass surgeries, respectively)

MetAP2 encodes a protein that functions at least in part by enzymatically removing the amino terminal methionine residue from certain newly translated proteins such as glyceraldehyde-3-phosphate dehydrogenase (Warder et al. (2008) J Proteome Res 7:4807). Increased expression of the MetAP2 gene has been historically associated with various forms of cancer. Molecules inhibiting the enzymatic activity of MetAP2 have been identified and have been explored for their utility in the treatment of various tumor types (Wang et al. (2003) Cancer Res. 63:7861) and infectious diseases such as microsporidiosis, leishmaniasis, and malaria (Zhang et al. (2002) J. Biomed. Sci. 9:34). However, such MetAP2 inhibitors may be useful as well for patients with excess adiposity and conditions related to adiposity including type 2 diabetes, hepatic steatosis, and cardiovascular disease (via e.g. ameliorating insulin resistance, reducing hepatic lipid content, and reducing cardiac workload). Methods of treating obese subjects that are more effective than e.g. dieting alone are clearly needed.

### SUMMARY

This disclosure generally relates to methods of treating an overweight or obese subject or patient that includes administering an effective amount of a METAP-2 inhibitor to a patient in need thereof, wherein the effective amount is proportional to excess body weight or excess adipose tissue of said patient. A patient may be a human or a companion animal such as a cat or a dog.

Provided herein, in an embodiment, is a method of treating obesity in a patient in need thereof, comprising administering an effective amount of a MetAP-2 inhibitor, wherein the effective amount is proportional to excess body weight or excess adipose tissue of said patient. For example, provided herein is a method of treating obesity in a patient in need thereof, comprising: determining the excess body weight or excess adipose tissue of said patient; determining an effective dose of a MetAP-2 based on the excess body weight or adipose tissue for said patient; and administering the effective dose to said patient.

In another embodiment, a method of treating obesity in a patient in need thereof is provided, comprising administering to a patient an effective amount of a MetAP-2 inhibitor, wherein the effective amount is capable of increasing adiponectin levels in the patient by at least 50% above the adiponectin level in the patient before administration of the inhibitor. For example, the effective amount may be capable of increasing adiponectin levels by 60% or more as compared to adiponectin levels in the patient before treatment, and/or the effective amount may reduce the ratio of leptin to adiponectin plasma levels in the patient after administration.

Also provided herein is a method of optimizing weight loss in a patient undergoing weight loss treatment, comprising administering an amount of a MetAP-2 inhibitor to said patient; determining the increase in adiponectin in said patient; increasing the amount of the MetAP-2 inhibitor administered to the patient if the change in adiponectin in the patient is less than an increase of about 60% or more as compared to the adiponectin level of the patient before administration of the MetAP-2 inhibitor. In an embodiment, a method of optimizing weight loss in a patient undergoing weight loss treatment is provided, comprising administering an amount of a MetAP-2 inhibitor to said patient; determining the increase in adiponectin in said patient; and increasing the amount of the MetAP-2 inhibitor administered to the patient if a reduction in the ratio of leptin to adiponectin in the plasma of the patient is not greater than 50%.

Contemplated MetAP-2 inhibitors for use in the disclosed methods include substantially irreversible inhibitors, e.g., aMetAP-2 inhibitor is selected from the group consisting of a fumagillin, fumagillol or fumagillin ketone, siRNA, shRNA, an antibody, or a antisense compound, or O-(4-dimethylaminoethoxycinnamoyl)fumagillol and pharmaceutically acceptable salts thereof. Reversible inhibitors are also contemplated.

Contemplated human patients may have an initial body mass index of at least about 27 kg/m², at least about 30 kg/m², or at least about 35, or at least about 40 kg/m². Administering as contemplated herein may comprise subcutaneous administration or intravenous administration.

For example, provided herein is a method for treating obesity in a patient having an initial body mass index of at least about 30 kg/m², comprising administering to the patient (e.g. human), a dose of a formulation comprising a therapeutically effective amount of a MEetAP-2 inhibitor, wherein the effective amount is proportional to excess body weight of the patient, as well as a method for reducing the weight of a patient (e.g. human) having an initial body mass index of at least about 27 kg/m², and suffering from weight related co-morbidity (e.g., hypertension, type 2 diabetes, dyslipidemia, and/or central adiposity), comprising administering to the patient, a dose of a formulation comprising a therapeutically effective amount of a MEetAP-2 inhibitor, wherein the effective amount is proportional to excess body weight of the patient. For example, the effective amount may be administered on a less than daily basis (e.g. twice weekly, weekly, or every 3 or 4 days).

### BRIEF DESCRIPTION OF FIGURES

FIG. 1 depicts body weight change (percent) with biweekly dosing of 0.1 mg/m², 0.3 mg/m², and 0.9 mg/m² (approximately 1.8 to 2.1 mg) of 6-O-(4-dimethylaminoethoxy)cinnamoyl fumagillol in human patients. Values are means±SEM (n=6 per dose level study); p values derived from 2-way ANOVA with Bonferroni post-test comparisons (*, p<0.05; **, p<0.01; ***, p<0.001 vs. placebo).

FIG. 2 depicts VAS reports of hunger decline by approximately 50% with body weight change (percent) with biweekly dosing of f 0.1 mg/m², 0.3 mg/m², and 0.9 mg/m² (approximately 1.8 to 2.1 mg) of 6-O-(4-dimethylaminoethoxy)cinnamoyl fumagillol in human patients. Values are means (n=6 per dose level) of changes in fasted Visual Analog Scale scores for each visit day vs. Day 1 for population; p values derived from 2-way ANOVA with Bonferroni post-test comparisons (*, p<0.05; **, p<0.01 vs. placebo).

FIG. 3 depicts dose exposure stability. Y-axis is plasma concentration of compound in ng/mL trials of participants receiving 0.9 mg/m².

FIG. 4 shows average pharmacokinetic (PK) profile with dose (ng/ml) of drug at day 26 of trials participants receiving 0.1, 0.3 and 0.9 mg/m².

FIG. 5 depicts correlation of weight change and exposure of drug at 24 hours.

FIG. 6 depicts the body weight change (kg) as a function of a μg per kg excess weight dose of 6-O-(4-dimethylaminoethoxy)cinnamoyl fumagillol in obese human patients. X axis is dose of compound (μg/kg of excessive weight).

FIG. 7 depicts weight change in patients administered a twice a week dose of 6-O-(4-dimethylaminoethoxy)cinnamoyl fumagillol, with varied dosage amounts. Weight loss plateaus at 0.9 mg/m² (˜1.8 mg dose) administered twice weekly, with a maximum efficacy obtained with a dose administered biweekly of ˜20 μg/kg.

FIG. 8 depicts the correlation between adiponectin changes and excess weight loss after administration of the disclosed MetAP-2 inhibitor in human obese patients.

FIG. 9 depicts the effect of administration of the disclosed MetAP-2 inhibitor on the ratio of leptin to adiponectin serum levels in human obese patients after administration.

### DETAILED DESCRIPTION

### Overview

The disclosure in part relates to treatment of e.g., obesity and arises in part due to the unexpected discovery that dosing and/or effectiveness of disclosed drugs depend on the amount of excess body weight of an overweight patient, rather than total body weight of the patient.

Obesity and being overweight refer to an excess of fat in proportion to lean body mass. Excess fat accumulation is associated with increase in size (hypertrophy) as well as number (hyperplasia) of adipose tissue cells. Obesity is variously measured in terms of absolute weight, weight:height ratio, degree of excess body fat, distribution of subcutaneous fat, and societal and esthetic norms. A common measure of body fat is Body Mass Index (BMI). The BMI refers to the ratio of body weight (expressed in kilograms) to the square of height (expressed in meters). Body mass index may be accurately calculated using the formulas: SI units: BMI=weight(kg)/(height²(m²), or US units: BMI=(weight(lb)*703)/(height²(in²).

In accordance with the U.S. Centers for Disease Control and Prevention (CDC), an overweight adult has a BMI of 25 kg/m² to 29.9 kg/m², and an obese adult has a BMI of 30 kg/m² or greater. A BMI of 40 kg/m² or greater is indicative of morbid obesity or extreme obesity. For children, the definitions of overweight and obese take into account age, stature, and gender as they relate to what are appropriate amounts of body fat and do not strictly rely on upon BMI calculations.

BMI does not account for the fact that excess adipose can occur selectively in different parts of the body, and development of adipose tissue can be more dangerous to health in some parts of the body rather than in other parts of the body. For example, “central obesity”, typically associated with an “apple-shaped” body, results from excess adiposity especially in the abdominal region, including belly fat and intra-abdominal or visceral fat, and carries higher risk of co-morbidity than “peripheral obesity”, which is typically associated with a “pear-shaped” body resulting from excess adiposity especially on the hips. Measurement of waist/hip circumference ratio (WHR) can be used as an indicator of central obesity. A minimum WHR indicative of central obesity has been variously set, and a centrally obese adult typically has a WHR of about 0.85 or greater if female and about 0.9 or greater if male. Methods of determining whether a subject is overweight or obese that account for the ratio of excess adipose tissue to lean body mass may involve obtaining a body composition of the subject. Body composition can be obtained by measuring the thickness of subcutaneous fat in multiple places on the body, such as the abdominal area, the subscapular region, arms, buttocks and thighs. These measurements are then used to estimate total body fat with a margin of error of approximately four percentage points. Another method is bioelectrical impedance analysis (BIA), which uses the resistance of electrical flow through the body to estimate body fat. Another method is using a large tank of water to measure body buoyancy. Increased body fat will result in greater buoyancy, while greater muscle mass will result in a tendency to sink. Yet another method is fan-beam dual energy X-ray absorptiometry (DEXA). DEXA allows body composition, particularly total body fat and/or regional fat mass, to be determined non-invasively.

Excess body weight may be assessed, for example, by comparing the weight of a patient in need of treatment to the weight of the same patient that would achieve a desired, e.g. non-obese, BMI (e.g. a desired BMI of about 25 or less). For example, excess body weight of a 1.6 m patient weighing 89.6 kg (and having a BMI of 35) may be found by calculating the weight required for a BMI of 25 (i.e., about 64 kg); the initial excess body weight of such patient would about 89.6−64=25.6 kg. Ideal body weight can be assessed, for example, by calculating 25*(height of patient)2, or e.g., by consulting Metropolitan or other life insurance tables.

### MetAP2 Inhibitors

MetAP2 inhibitors refer to a class of molecules that inhibit or modulate the activity of MetAP2, e.g., the ability of MetAP2 to cleave the N-terminal methionine residue of newly synthesized proteins to produce the active form of the protein, or the ability of MetAP2 to regulate protein synthesis by protecting the subunit of eukaryotic initiation factor-2 (eIF2) from phosphorylation.

Exemplary MetAP2 inhibitors may include irreversible inhibitors that covalently bind to MetAP2. For example, such irreversible inhibitors include fumagillin, fumagillol, and fumagillin ketone.

Derivatives and analogs of fumagillin, and pharmaceutically acceptable salts thereof are contemplated herein as irreversible MetAP2 inhibitors, such as O-(4-dimethylaminoethoxycinnamoyl)fumagillol (also referred to herein as Compound A), O-(3,4,5-trimethoxycinnamoyl)fumagillol, O-(4-chlorocinnamoyl)fumagillol; O-(4-aminocinnamoyl)fumagillol; O-(4-dimethylaminoethoxycinnamoyl)fumagillol; O-(4-methoxycinnamoyl)fumagillol; O-(4-dimethylaminocinnamoyl)fumagillol; O-(4-hydroxycinnamoyl)fumagillol; O-(3,4-dimethoxycinnamoyl)fumagillol; O-(3,4-methylenedioxycinnamoyl)fumagillol; O-(3,4,5-trimethoxycinnamoyl)fumagillol; O-(4-nitrocinnamoyl)fumagillol; O-(3,4-dimethoxy-6-aminocinnamoyl)fumagillol; O-(4-acetoxy-3,5-dimethoxycinnamoyl)fumagillol; O-(4-ethylaminocinnamoyl)fumagillol; O-(4-ethylaminoethoxycinnamoyl)fumagillol; O-(3-dimethylaminomethyl-4-methoxycinnamoyl)fumagillol; O-(4-trifluoromethylcinnamoyl)fumagillol; O-(3,4-dimethoxy-6-nitrocinnamoyl)fumagillol; O-(4-acetoxycinnamoyl)fumagillol; O-(4-cyanocinnamoyl)fumagillol; 4-(4-methoxycinnamoyl)oxy-2-(1,2-epoxy-1,5-dimethyl-4-hexenyl)-3-methoxy-1-chloromethyl-1-cyclohexanol; O-(3,4,5-trimethoxycinnamoyl)fumagillol; O-(4-dimethylaminocinnamoyl)fumagillol; O-(3,4,5-trimethoxycinnamoyl)oxy-2-(1,2-epoxy-1,5-dimethyl-4-hexenyl)-3-m-ethoxy-1-chloromethyl-1-cyclohexanol; O-(4-dimethylaminocinnamoyl)oxy-2-(1,2-epoxy-1,5-dimethyl-4-hexenyl)-3-me-thoxy-1-chloromethyl-1-cyclohexanol; O-(3,5-dimethoxy-4-hydroxycinnamoyl)fumagillol or O-(chloracetyl-carbamoyl)fumagillol (TNP-470), and/or pharmaceutically acceptable salts thereof (e.g. O-(4-dimethylaminoethoxycinnamoyl)fumagillol oxalate).

Fumagillin, and some derivatives thereof, have a carboxylic acid moiety and can be administered in the form of the free acid. Alternatively, contemplated herein are pharmaceutically acceptable salts of fumagillin, fumagillol, and derivatives thereof.

Pharmaceutically acceptable salts illustratively include those that can be made using the following bases: ammonia, L-arginine, benethamine, benzathene, betaine, bismuth, calcium hydroxide, choline, deanol, diethanolamine, diethylamine, 2-(diethylamino)ethanol, ethylenediamine, N-methylglucamine, hydrabamine, 1H-imidazole, lysine, magnesium hydroxide, 4-(2-hydroxyethyl)morpholine, piperazine, potassium hydroxide, 1-(2-hydroxyethyl)pyrrolidine, sodium hydroxide, triethanolamine, zinc hydroxide, diclyclohexlamine, or any other electron pair donor (as described in Handbook of Pharmaceutical Salts, Stan & Wermuth, VHCA and Wiley, Uchsenfurt-Hohestadt Germany, 2002). Contemplated pharmaceutically acceptable salts may include hydrochloric acid, bromic acid, sulfuric acid, phosphoric acid, nitric acid, formic acid, acetic acid, trifluoroacetic acid, oxalic acid, fumaric acid, tartaric acid, maleic acid, methanesulfonic acid, benzenesulfonic acid or para-toluenesulfonic acid.

Esters of the present invention may be prepared by reacting e.g. fumagillin or fumagillol with the appropriate acid under standard esterification conditions described in the literature (Houben-Weyl 4th Ed. 1952, Methods of Organic Synthesis). Suitable fumagillin esters include ethyl methanoate, ethyl ethanoate, ethyl propanoate, propyl methanoate, propyl ethanoate, and methyl butanoate.

In another embodiment, contemplated irreversible inhibitors of MetAP2 may include a siRNA, shRNA, an antibody or an antisense compound of MetAP2.

Further examples of reversible and irreversible MetAP2 inhibitors are provided in the following references, each of which is hereby incorporated by reference: Olson et al. (U.S. Pat. No. 7,084,108 and WO 2002/042295), Olson et al. (U.S. Pat. No. 6,548,477; U.S. Pat. No. 7,037,890; U.S. Pat. No. 7,084,108; U.S. Pat. No. 7,268,111; and WO 2002/042295), Olson et al. (WO 2005/066197), Hong et al. (U.S. Pat. No. 6,040,337)., Hong et al. (U.S. Pat. No. 6,063,812 and WO 1999/059986), Lee et al. (WO 2006/080591), Kishimoto et al. (U.S. Pat. No. 5,166,172; U.S. Pat. No. 5,698,586; U.S. Pat. Nos. 5,164,410; and 5,180,738), Kishimoto et al. (U.S. Pat. No. 5,180,735), Kishimoto et al. (U.S. Pat. No. 5,288,722), Kishimoto et al. (U.S. Pat. No. 5,204,345), Kishimoto et al. (U.S. Pat. No. 5,422,363), Liu et al. (U.S. Pat. No. 6,207,704; U.S. Pat. No. 6,566,541; and WO 1998/056372), Craig et al. (WO 1999/057097), Craig et al. (U.S. Pat. No. 6,242,494), BaMaung et al. (U.S. Pat. No. 7,030,262), Comess et al. (WO 2004/033419), Comess et al. (US 2004/0157836), Comess et al. (US 2004/0167128), Henkin et al. (WO 2002/083065), Craig et al. (U.S. Pat. No. 6,887,863), Craig et al. (US 2002/0002152), Sheppard et al. (2004, Bioorganic & Medicinal Chemistry Letters 14:865-868), Wang et al. (2003, Cancer Research 63:7861-7869), Wang et al. (2007, Bioorganic & Medicinal Chemistry Letters 17:2817-2822), Kawai et al. (2006, Bioorganic & Medicinal Chemistry Letters 16:3574-3577), Henkin et al. (WO 2002/026782), Nan et al. (US 2005/0113420), Luo et al. (2003, J. Med. Chem., 46:2632-2640), Vedantham et al. (2008, J. Comb. Chem., 10:195-203), Wang et al. (2008, J. Med. Chem., 51 (19):6110-20), Ma et al. (2007, BMC Structural Biology, 7:84) and Huang et al. (2007, J. Med. Chem., 50:5735-5742), Evdokimov et al. (2007, PROTEINS: Structure, Function, and Bioinformatics, 66:538-546), Garrabrant et al. (2004, Angiogenesis 7:91-96), Kim et al. (2004, Cancer Research, 64:2984-2987), Towbin et al. (2003, The Journal of Biological Chemistry, 278(52):52964-52971), Marino Jr. (U.S. Pat. No. 7,304,082), Kallender et al. (U.S. patent application number 2004/0192914), and Kallender et al. (U.S. patent application numbers 2003/0220371 and 2005/0004116). Other MetAP2 inhibitors contemplated herein are disclosed in U.S. Ser. No. 61/310,776; 61/293,318; 61/366,650 and PCT/US10/52050 (all of the above are hereby incorporated by reference in their entirety).

For example, contemplated MetAP2 inhibitors may include:

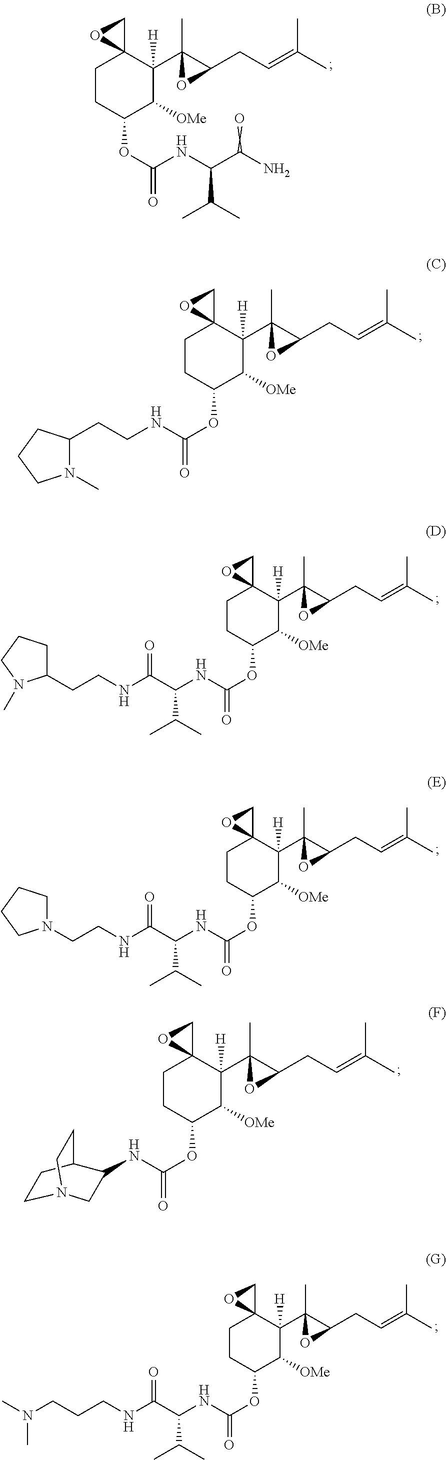

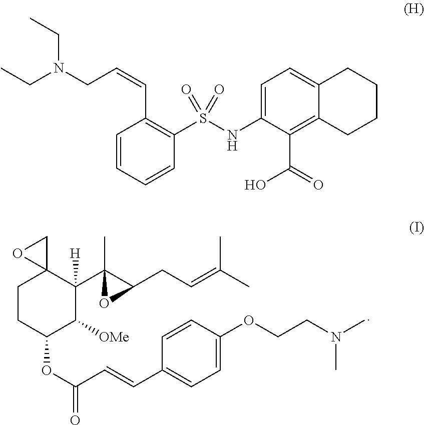 [SMILES: CCN(CC)C/C=C\c1ccccc1S(=O)(=O)Nc1ccc2c(c1C(=O)O)CCCC2 ; CO[C@@H]1[C@H](OC(=O)/C=C/c2ccc(OCCN(C)C)cc2)CCC2(CO2)[C@H]1[C@@]1(C)O[C@@H]1CC=C(C)C]

### Methods

A method for treating obesity or for reducing body weight in a patient in need thereof is provided herein, comprising administering to the patient, on a less than daily basis, a dose of a formulation comprising a therapeutically effective amount of a compound 6-O-(4-dimethylaminoethoxy)cinnamoyl fumagillol or pharmaceutically acceptable salts thereof. Such methods may include administering to the patient a single dose of the formulation about every other day (e.g., every two days), twice weekly (e.g. every 3 days, every 4 days, every 5 days, every 6 days or e.g. administered with an interval of about 2 to about 3 days between doses), once a week, every other week, twice monthly, once a month or even less often. It may be appreciated that methods that include administering a single dose on a less frequent basis, may, in some embodiments, be a method directed to maintaining a specific weight, such as a more optimal body weight after treatment using other methods disclosed herein. Disclosed methods may include administering a dose of a disclosed compound on a less than daily basis until a desired weight is achieved.

In another embodiment, provided herein is a method for treating obesity or for reducing body weight in a patient in need thereof, comprising administering to the patient a dose of a formulation comprising a therapeutically effective amount of a compound 6-O-(4-dimethylaminoethoxy)cinnamoyl fumagillol or pharmaceutically acceptable salts thereof, for a first period of time, withheld for a second period of time, and again optionally administered for a third period of time, e.g., alternate dosing regimens. For example, for the first period of time a patient may be administered a disclosed formulation daily, every other day, every three, four or five days, biweekly, monthly, or yearly; during the second period of time (e.g. 1 day, 1 week, 2 weeks, 1 month) no dose is administered; and during e.g. a third period of time, the patient may be administered on a regimen similar or different to the first period of time, for example, every other day, every three, four or five days, biweekly, monthly, or yearly. At each administration or period time, the route of administration may be different or the same as another period of time.

The therapeutically effective amount administered in the disclosed methods such as those above may provide a patient with an initial weight loss of about 0.3% to about 2%, about 0.4% to about 2%, or about 0.5% to about 2% or more, or about 0.5 kg to about 2 kg or more of the initial patient weight even after an initial dose, or after administration of two doses, or after administering after an first period of time, e.g., such methods may incur weight loss for three or four days or more after administration (e.g. parenteral (for example intravenous) administration) of a single dose. For example, a patient, after receiving a first dose and/or after receiving a subsequent dose, may continue to lose weight for three or four days or more without further administration of a disclosed compound. In some embodiments, administration of an initial first dose, or administration of a first and second dose (e.g., both administered in the same week), may provide about 0.5 kg to about 2 kg or more of weight loss. Subsequent administration may result in further weight loss, until a target patient weight is achieved.

Therapeutically effective doses may be calculated, for example, on the basis of body surface area (BSA), which can be determined using formulae such as those described by Mosteller (Mosteller R D, N Engl J Med 1987 Oct. 22; 317(17):1098), in which BSA is calculated in SI units as BSA (m²)=([Height(cm)×Weight(kg)]/3600)/2 (e.g. BSA=SQRT((cm*kg)/3600)), or US units, in which BSA (m²)=([Height(in)×Weight(lbs)]/3131)^{1/2}. In some embodiments, the therapeutically effective amount administered (e.g., intravenously) to patient using a disclosed method is about 0.5 mg/m² to about 3 mg/m², or about 0.9 mg/m² (or approximately 10 to 20 μg per kilo of total body weight) or more of a disclosed compound. In other embodiments, a therapeutically effective amount is based on excess body weight (or excess adipose tissue), for example, at least about 30 μg of a disclosed compound per kg of excess adipose tissue, (or excess body weight) of the patient, or least about 40 μg per kg or more of excess adipose tissue, (or excess body weight) of the patient, e.g., about 30 μg per kg of excess adipose tissue (or excess body weight) to about 60 μg per kg, about 40 μg per kg to about 60 μg per kg, or about 35 μg per kg to about 45 μg per kg, or about 35 μg per kg to about 50 μg per kg, or about 35 μg per kg to about 80 μg per kg of excess adipose tissue (or excess body weight).

For example, provided herein is a method for treating obesity or for reducing body weight in a patient in need thereof, comprising administering to the patient a dose comprising about 0.9 mg/m² (e.g. of calculated surface area) or more (or e.g., about 0.75 mg/m² to about 1.5 mg/m², or about 0.75 mg/m² to about 3 mg/m², or about 0.9 mg/m² to about 3 mg/m², or about 0.9 mg/m² to about 1.5 mg/m²) (e.g., administered intravenously) of a compound 6-O-(4-dimethylaminoethoxy)cinnamoyl fumagillol or pharmaceutically acceptable salts thereof, wherein a single administration of the dose reduces weight in the patient for at least four days (e.g. administered intravenously) of a compound 6-O-(4-dimethylaminoethoxy)cinnamoyl fumagillol or pharmaceutically acceptable salts thereof, wherein a single administration of the dose reduces weight in the patient for at least four days. Contemplated doses may include therapeutically effective amount of 6-O-(4-dimethylaminoethoxy)cinnamoyl fumagillol or pharmaceutically acceptable salts thereof with, e.g., about 0.5 mg/m² to about 3 mg/m² (based on a patient's actual or calculated surface area), e.g. about 0.9 mg/m² to about 1.5 mg/m², 0.9 mg/m² to about 3 mg/m²e.g., about 1.25, 1.5, 2, 2.5 or 3 mg/m². In other embodiments, a therapeutically effective amount of a disclosed compound may be at least about 20 to about 80 μg per kg, or at least about of 20 to about 40 μg per kg excess body weight of the patient. For example, a therapeutically effective amount of a disclosed compound, e.g., administered every three or four days may be a dosage that includes e.g., about 1.8 mg, 2.5 mg, 2 mg, 3 mg, 4 mg, 5 mg, or 6 mg of a disclosed compound. Administration of a single dose with such a method may reduce weight in the patient for at least three or four days, or up to seven days, for example, even without further administration.

In another embodiment, a method of treating obesity in a patient in need thereof is provided, comprising administering an effective amount of a MetAP-2 inhibitor, wherein the effective amount is proportional to excess body weight of said patient. For example, in some embodiments, such effective amount may not be proportional to total body weight. A method of treating obesity in a patient in need thereof is also provided, comprising determining the excess body weight or excess adipose tissue of said patient; determining an effective dose of a MetAP-2 based on the excess body weight or adipose tissue for said patient; and administering the effective dose to said patient.

For example, provided herein is a method for treating obesity or for reducing body weight, comprising administering to a patient in need thereof at least about 20 μg, 30 μg, or at least about 40 μg or more per kilogram of excess body weight (e.g., about 20 μg to about 80 μg, or about 40 μg to about 60 μg per kilogram of excess body weight) of 6-O-(4-dimethylaminoethoxy)cinnamoyl fumagillol or pharmaceutically acceptable salts thereof per kg of excess adipose tissue (or excess body weight) of the patient. Disclosed methods may reduce the body weight of the patient for at least 3 or 4 days after administration, or at least 7 days after administration, at least 14 days after administration, or even at least 1 month after administration, e.g., without further administration of the compound during that time. It is understood that even though an administration may provide for weight loss e.g. at least for 3 or 4 days, a disclosed compound may be administered more frequently, e.g. every other day.

In another embodiment, a method of treating obesity or for reducing body weight is provided comprising administering a dose, on a less than weekly basis, of 6-O-(4-dimethylaminoethoxy)cinnamoyl fumagillol or pharmaceutically acceptable salts wherein the doses comprises at least about 35 μg to about 75 μg per kilogram, about 20 to about 60 μg/kg ideal body weight, or about 20 to about 40 μg/kg of the compound per ideal body weight of the patient, wherein ideal body weight is 25*(patient height in meters). For example, a therapeutically effective amount of a disclosed compound, e.g., administered every three or four days may be a dosage that includes e.g., about 1.8 mg, 2.5 mg, 2 mg, 3 mg, 4 mg, 5 mg, or 6 mg of a disclosed compound.

For example, provided herein is a method of reducing the body weight of a patient in need thereof for at least about three or at least about four days, comprising administering (e.g. parenterally administering) to the patient a single dose of at least about 30 μg of 6-O-(4-dimethylaminoethoxy)cinnamoyl fumagillol or pharmaceutically acceptable salts thereof, per kg of excess adipose tissue of the patient. In some embodiments, such methods may further include administering a second dose of at least about 30 μg of 6-O-(4-dimethylaminoethoxy)cinnamoyl fumagillol or pharmaceutically acceptable salts thereof, per kg of excess body weight of the patient or, in other embodiments, about 0.9 mg/m² or more (or about 0.75 mg/m² to about 3 mg/m²) of the compound at least about 4 days after administration of the single dose. A second dose, for example, may be administered at intervals of e.g., three or four days or more. In another embodiments, disclosed methods may further include administering subsequent doses of a MetAP2 inhibitor (e.g. —O-(4-dimethylaminoethoxy)cinnamoyl fumagillol) at intervals of between about 4 days and 1 month. Such disclosed methods may, upon administration, increase the adiponectin levels of the patient by at least 50% above, or at least 20%, at least 30% at least 40%, or more, e.g. at least about 20% to about 60% above the adiponectin level in the patient before administration (e.g. baseline adiponectin of the patient before treatment.)

In another aspect, a method of treating obesity in a patient in need thereof is provided comprising administering to a patient an effective amount of an MetAP-2 inhibitor, wherein the effective amount is capable of increasing adiponectin levels in the patient by at least 50% above, at least 60% above, or at least 20%, at least 30% at least 40%, or more, e.g. at least about 20% to about 60%, or the adiponectin level in the patient before administration of the inhibitor. Such an effective amount of the MetAP-2 inhibitor may reduce the ratio of leptin to adiponectin plasma levels in the patient after administration. Further, therapeutically effective doses contemplated herein will not typically induce any clinically significant anti-angiogenic action.

Also provided herein is a method of optimizing weight loss in a patient undergoing weight loss treatment, comprising a) administering an amount of a MetAP-2 inhibitor to said patient; b) determining the increase in adiponectin in said patient; and c) increasing the amount of the MetAP-2 inhibitor administered to the patient if the change in adiponectin in the patient is less than an increase of about 60% or more (or 50% or more, e.g. 30% to about 60% as compared to the adiponectin level of the patient before administration of the MetAP-2 inhibitor.

In another embodiment, a method of optimizing weight loss in a patient undergoing weight loss treatment, comprising a) administering an amount of a MetAP-2 inhibitor to said patient; b) determining the increase in adiponectin in said patient; c) increasing the amount of the MetAP-2 inhibitor administered to the patient if a reduction in the ratio of leptin to adiponectin in the plasma of the patient is not greater than 50%, or not greater than 40%, e.g., the reduction of the ratio of leptin to adiponection is reduced by about 10%, 20%, 30% or 40%.

In addition to being overweight or obese, a patient may be suffering from other overweight- or obesity-related co-morbidities, i.e., diseases and other adverse health conditions associated with, exacerbated by, or precipitated by being overweight or obese. Because being overweight or obese is associated with other adverse health conditions or co-morbidities, for example diabetes, administering MetAP2 inhibitors may bring a benefit in ameliorating, arresting development of or, in some cases, even eliminating, these overweight- or obesity-related conditions or co-morbidities. For example, contemplated methods of reducing body weight disclosed herein includes treatment those patients who have e.g., a BMI of greater than 27 kg/m², and who have one or more weight related co-morbidities, such as hypertension, type 2 diabetes, dyslipidemia, and/or central adiposity.

In some embodiments, methods provided herein may further include administering at least one other agent in addition to a MetAP2 inhibitor, e.g., an agent directed to treatment of these overweight- or obesity-related conditions.

Contemplated other agents include those administered to treat type 2 diabetes such as sulfonylureas (e.g., chlorpropamide, glipizide, glyburide, glimepiride); meglitinides (e.g., repaglinide and nateglinide); biguanides (e.g., metformin); thiazolidinediones (rosiglitazone, troglitazone, and pioglitazone); glucagon-like 1 peptide mimetics (e.g. exenatide and liraglutide); sodium-glucose cotransporter inhibitors (e.g., dapagliflozin), renin inhibitors, and alpha-glucosidase inhibitors (e.g., acarbose and meglitol), and/or those administered to treat cardiac disorders and conditions, such hypertension, dyslipidemia, ischemic heart disease, cardiomyopathy, cardiac infarction, stroke, venous thromboembolic disease and pulmonary hypertension, which have been linked to overweight or obesity, for example, chlorthalidone; hydrochlorothiazide; indapamide, metolazone; loop diuretics (e.g., bumetanide, ethacrynic acid, furosemide, lasix, torsemide); potassium-sparing agents (e.g., amiloride hydrochloride, spironolactone, and triamterene); peripheral agents (e.g., reserpine); central alpha-agonists (e.g., clonidine hydrochloride, guanabenz acetate, guanfacine hydrochloride, and methyldopa); alpha-blockers (e.g., doxazosin mesylate, prazosin hydrochloride, and terazosin hydrochloride); beta-blockers (e.g., acebutolol, atenolol, betaxolol, nisoprolol fumarate, carteolol hydrochloride, metoprolol tartrate, metoprolol succinate, Nadolol, penbutolol sulfate, pindolol, propranolol hydrochloride, and timolol maleate); combined alpha- and beta-blockers (e.g., carvedilol and labetalol hydrochloride); direct vasodilators (e.g., hydralazine hydrochloride and minoxidil); calcium antagonists (e.g., diltiazem hydrochloride and verapamil hydrochloride); dihydropyridines (e.g., amlodipine besylate, felodipine, isradipine, nicardipine, nifedipine, and nisoldipine); ACE inhibitors (benazepril hydrochloride, captopril, enalapril maleate, fosinopril sodium, lisinopril, moexipril, quinapril hydrochloride, ramipril, trandolapril); angiotensin II receptor blockers (e.g., losartan potassium, valsartan, and Irbesartan); and combinations thereof, as well as statins such as mevastatin, lovastatin, pravastatin, simvastatin, velostatin, dihydrocompactin, fluvastatin, atorvastatin, dalvastatin, carvastatin, crilvastatin, bevastatin, cefvastatin, rosuvastatin, pitavastatin, and glenvastatin., typically for treatment of dyslipidemia.

Other agents that may be co-administered (e.g. sequentially or simultaneously) include agents administered to treat ischemic heart disease including statins, nitrates (e.g., Isosorbide Dinitrate and Isosorbide Mononitrate), beta-blockers, and calcium channel antagonists, agents administered to treat cardiomyopathy including inotropic agents (e.g., Digoxin), diuretics (e.g., Furosemide), ACE inhibitors, calcium antagonists, anti-arrhythmic agents (e.g., Sotolol, Amiodarone and Disopyramide), and beta-blockers, agents administered to treat cardiac infarction including ACE inhibitors, Angiotensin II receptor blockers, direct vasodilators, beta blockers, anti-arrhythmic agents and thrombolytic agents (e.g., Alteplase, Retaplase, Tenecteplase, Anistreplase, and Urokinase), agents administered to treat strokes including anti-platelet agents (e.g., Aspirin, Clopidogrel, Dipyridamole, and Ticlopidine), anticoagulant agents (e.g., Heparin), and thrombolytic agents, agents administered to treat venous thromboembolic disease including anti-platelet agents, anticoagulant agents, and thrombolytic agents, agents administered to treat pulmonary hypertension include inotropic agents, anticoagulant agents, diuretics, potassium (e.g., K-dur), vasodilators (e.g., Nifedipine and Diltiazem), Bosentan, Epoprostenol, and Sildenafil, agents administered to treat asthma include bronchodilators, anti-inflammatory agents, leukotriene blockers, and anti-Ige agents. Particular asthma agents include Zafirlukast, Flunisolide, Triamcinolone, Beclomethasone, Terbutaline, Fluticasone, Formoterol, Beclomethasone, Salmeterol, Theophylline, and Xopenex, agents administered to treat sleep apnea include Modafinil and amphetamines, agents administered to treat nonalcoholic fatty liver disease include antioxidants (e.g., Vitamins E and C), insulin sensitizers (Metformin, Pioglitazone, Rosiglitazone, and Betaine), hepatoprotectants, and lipid-lowering agents, agents administered to treat osteoarthritis of weight-bearing joints include Acetaminophen, non-steroidal anti-inflammatory agents (e.g., Ibuprofen, Etodolac, Oxaprozin, Naproxen, Diclofenac, and Nabumetone), COX-2 inhibitors (e.g., Celecoxib), steroids, supplements (e.g. glucosamine and chondroitin sulfate), and artificial joint fluid, agents administered to treat Prader-Willi Syndrome include human growth hormone (HGH), somatropin, and weight loss agents (e.g., Orlistat, Sibutramine, Methamphetamine, Ionamin, Phentermine, Bupropion, Diethylpropion, Phendimetrazine, Benzphetermine, and Topamax), agents administered to treat polycystic ovary syndrome include insulin-sensitizers, combinations of synthetic estrogen and progesterone, Spironolactone, Eflornithine, and Clomiphene, agents administered to treat erectile dysfunction include phosphodiesterase inhibitors (e.g., Tadalafil, Sildenafil citrate, and Vardenafil), prostaglandin E analogs (e.g., Alprostadil), alkaloids (e.g., Yohimbine), and testosterone, agents administered to treat infertility include Clomiphene, Clomiphene citrate, Bromocriptine, Gonadotropin-releasing Hormone (GnRH), GnRH agonist, GnRH antagonist, Tamoxifen/nolvadex, gonadotropins, Human Chorionic Gonadotropin (HCG), Human Menopausal Gonadotropin (HmG), progesterone, recombinant follicle stimulating hormone (FSH), Urofollitropin, Heparin, Follitropin alfa, and Follitropin beta, agents administered to treat obstetric complications include Bupivacaine hydrochloride, Dinoprostone PGE2, Meperidine HCl, Ferro-folic-500/iberet-folic-500, Meperidine, Methylergonovine maleate, Ropivacaine HCl, Nalbuphine HCl, Oxymorphone HCl, Oxytocin, Dinoprostone, Ritodrine, Scopolamine hydrobromide, Sufentanil citrate, and Oxytocic, agents administered to treat depression include serotonin reuptake inhibitors (e.g., Fluoxetine, Escitalopram, Citalopram, Paroxetine, Sertraline, and Venlafaxine); tricyclic antidepressants (e.g., Amitriptyline, Amoxapine, Clomipramine, Desipramine, Dosulepin hydrochloride, Doxepin, Imipramine, Iprindole, Lofepramine, Nortriptyline, Opipramol, Protriptyline, and Trimipramine); monoamine oxidase inhibitors (e.g., Isocarboxazid, Moclobemide, Phenelzine, Tranylcypromine, Selegiline, Rasagiline, Nialamide, Iproniazid, Iproclozide, Toloxatone, Linezolid, Dienolide kavapyrone desmethoxyyangonin, and Dextroamphetamine); psychostimulants (e.g., Amphetamine, Methamphetamine, Methylphenidate, and Arecoline); antipsychotics (e.g., Butyrophenones, Phenothiazines, Thioxanthenes, Clozapine, Olanzapine, Risperidone, Quetiapine, Ziprasidone, Amisulpride, Paliperidone, Symbyax, Tetrabenazine, and Cannabidiol); and mood stabilizers (e.g., Lithium carbonate, Valproic acid, Divalproex sodium, Sodium valproate, Lamotrigine, Carbamazepine, Gabapentin, Oxcarbazepine, and Topiramate), agents administered to treat anxiety include serotonin reuptake inhibitors, mood stabilizers, benzodiazepines (e.g., Alprazolam, Clonazepam, Diazepam, and Lorazepam), tricyclic antidepressants, monoamine oxidase inhibitors, and beta-blockers, and other weight loss agents, including serotonin and noradrenergic re-uptake inhibitors; noradrenergic re-uptake inhibitors; selective serotonin re-uptake inhibitors; and intestinal lipase inhibitors. Particular weight loss agents include orlistat, sibutramine, methamphetamine, ionamin, phentermine, bupropion, diethylpropion, phendimetrazine, benzphetermine, and topamax.

In some embodiments, contemplated methods may further comprise assessing one or more indices of on-going weight loss, e.g. the ketone body production level in a patient; and optionally adjusting the amount administered, thereby optimizing the therapeutic efficacy of the MetAP2 inhibitor.

In some embodiments, contemplated methods may further comprising assessing one or more indices of on-going weight loss, e.g. the ketone body production level in a patient; and optionally adjusting the amount administered; thereby optimizing the therapeutic efficacy of said MetAP2 inhibitor. Contemplated administration of Met-AP2 inhibitors in the disclosed methods include subcutaneous or intravenous administration. For example, injectable preparations are contemplated herein, for example, sterile injectable aqueous or oleaginous suspensions may be formulated according to the known art using suitable dispersing or wetting agents and suspending agents.

Disclosed or contemplated treatment regimens can include a corrective phase, during which a MetAP2 inhibitor dose sufficient to provide reduction of excess adiposity is administered, followed by a maintenance phase, during which a lower or equivalent MetAP2 inhibitor dose sufficient to prevent re-development of excess adiposity may be administered.

### EXAMPLES

The examples which follow are intended in no way to limit the scope of this invention but are provided to illustrate aspects of the disclosed methods. Many other embodiments of this invention will be apparent to one skilled in the art.

### Example 1

### Less than Weekly Administration of a MetAP2 Inhibitor to Obese Humans

Obese patients were treated in three cohorts with intravenous administration of a formulation of the compound 6-O-(4-dimethylaminoethoxy)cinnamoyl fumagillol oxalate. The compound was intravenously administered to each patient of a cohort (except for a placebo cohort) twice weekly for at least 26 days. Each of patients in the three non-placebo cohorts received either 0.1 mg/m² (cohort 1); 0.3 mg/m² (cohort 2); or 0.9 mg/m² (cohort 3) doses of the compound at the time of administration. The trial was conducted under the appropriate government and medical supervision.

Weight reduction occurred for 4 days after single administration of 0.9 mg/m2 dose, despite terminal half life of the drug of only 5.4 hours (T_{1/2} (H) λz (terminal) is 5.41±2.82 in cohort 3), indicating daily administration is not needed. Table 1 summarizes body weight determination of the 9 patients in the 0.9 mg/m² cohort on the day of the first dose and prior to a second dose administered four days later.

TABLE 1

| Subject | Starting | Ending | Weight | Weight |
|---|---|---|---|---|
| No. | weight (kg) | weight (kg) | Change (kg) | Change (%) |
| 1 | 96.3 | 95.7 | −0.6 | −0.6 |
| 2 | 101.5 | 100.4 | −1.1 | −1.1 |
| 3 | 104.6 | 103.7 | −0.9 | −0.9 |
| 4 | 102.4 | 100.5 | −1.9 | −1.9 |
| 5 | 108.6 | 107.5 | −1.1 | −1.0 |
| 6 | 107.9 | 107.9 | 0 | 0.0 |
| 7 | 105.5 | 105 | −0.5 | −0.5 |
| 8 | 118.7 | 118 | −0.7 | −0.6 |
| 9 | 96 | 96.2 | 0.2 | 0.2 |
| Average | 104.6 | 103.9 | −0.7 | −0.7 |
| SEM | 2.3 | 2.3 | 0.0 | 0.2 |
| p | | | 0.004 | 0.004 |

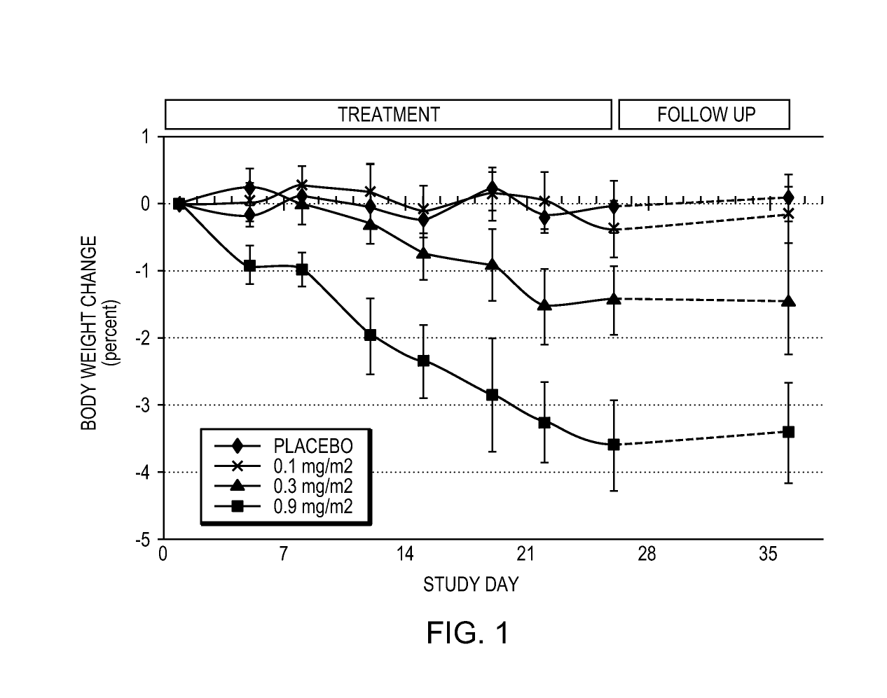

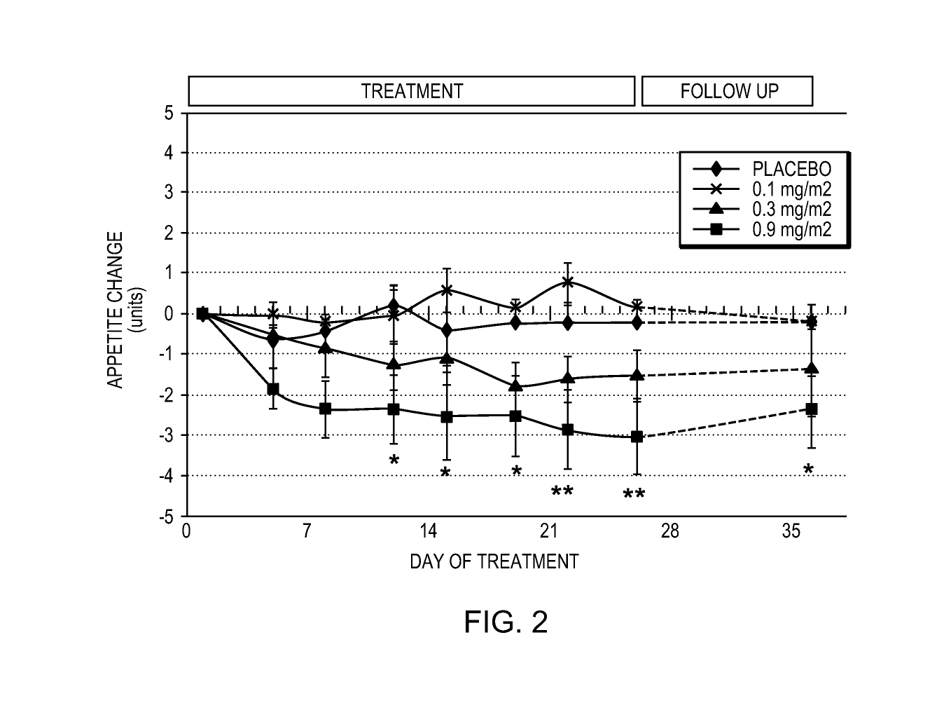

Of the 9 subjects treated at the 0.9 mg/m² dose level, 7 patients showed greater than 3-4 percent body weight loss over 26 days of exposure. FIG. 1 depicts the body weight change (loss) as a percent before each administration of the biweekly dose for the 0.9 mg/m² cohort. FIG. 2 depicts the Visual Analog Scale reports of hunger decline about 50% following successive biweekly dose administration for the 0.9 mg/m² cohort. As shown in FIGS. 1 and 2, a follow up check at day 36 was conducted; patients were weighed again at this 36 day mark and the weight loss from the twice weekly regimen over 26 days was substantially sustained.

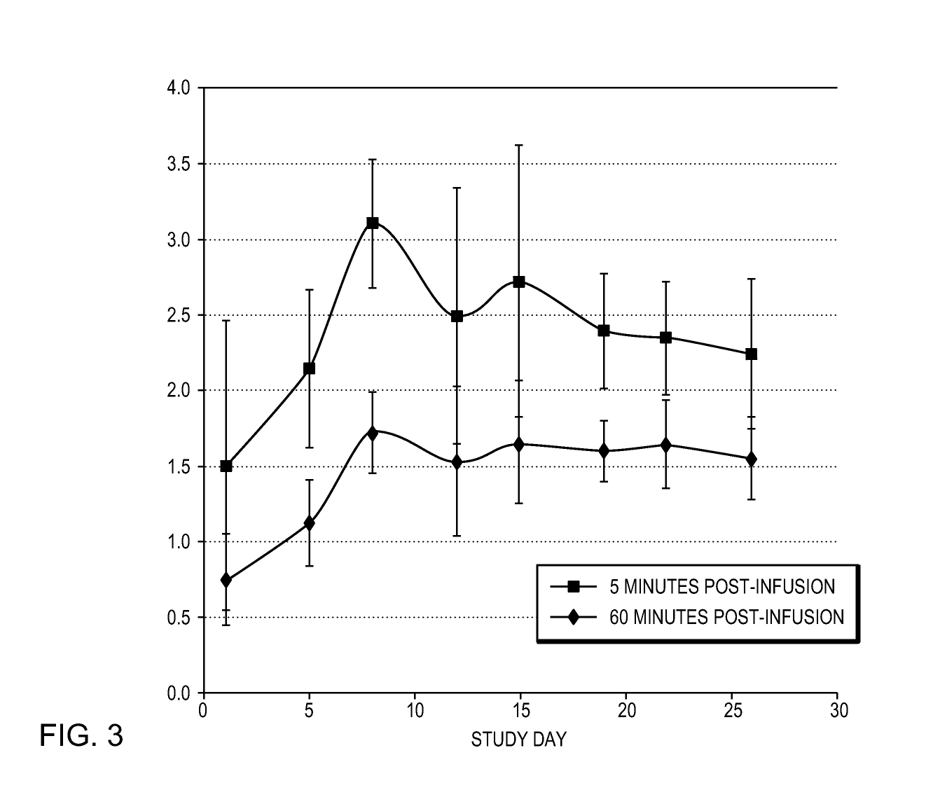

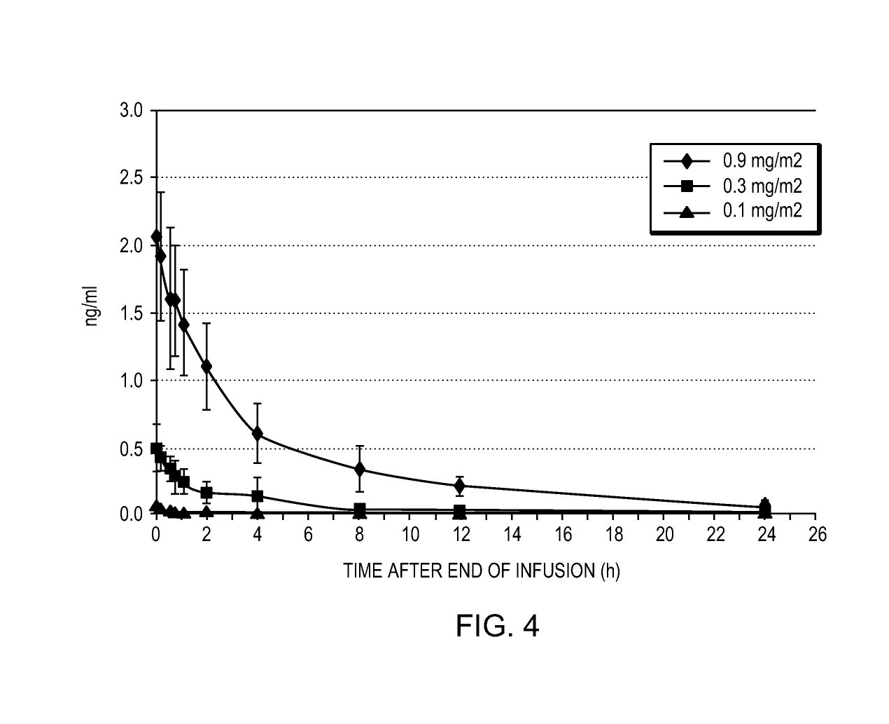

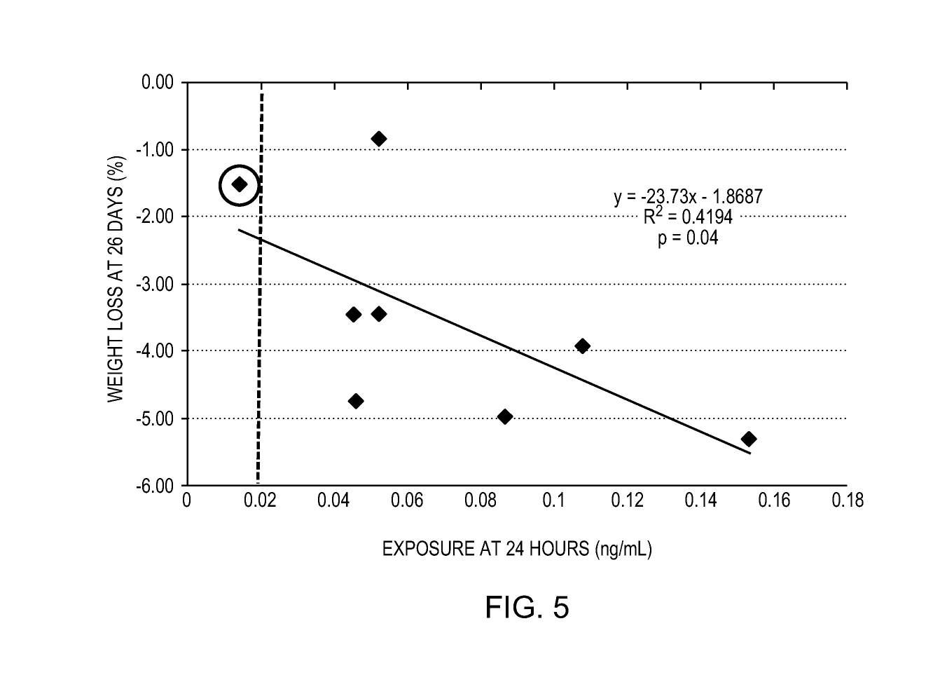

FIG. 3 depicts exposure of drug as function of dose with measurements taken at 5 min and 60 min post dose, and shows that drug exposure stabilizes by the third or fourth dose administered. There was a significant correlation between weight loss of patients and maintained exposure of 0.02 ng/mL of the drug above the level of quantitation of the assay 24 hours after dosing, as shown in FIG. 4 (circled data point is exposure of one poor responder below the level of quantitation of assay). The PK profile at day 26 is representative of steady state (see FIG. 5). Lack of efficacy at 0.1 and 0.3 mg/m² supports maintaining drug exposure above 0.02 ng/mL at 24 hours post dose.

### Example 2

### Correlation of Effective Dose and Excess Body Weight

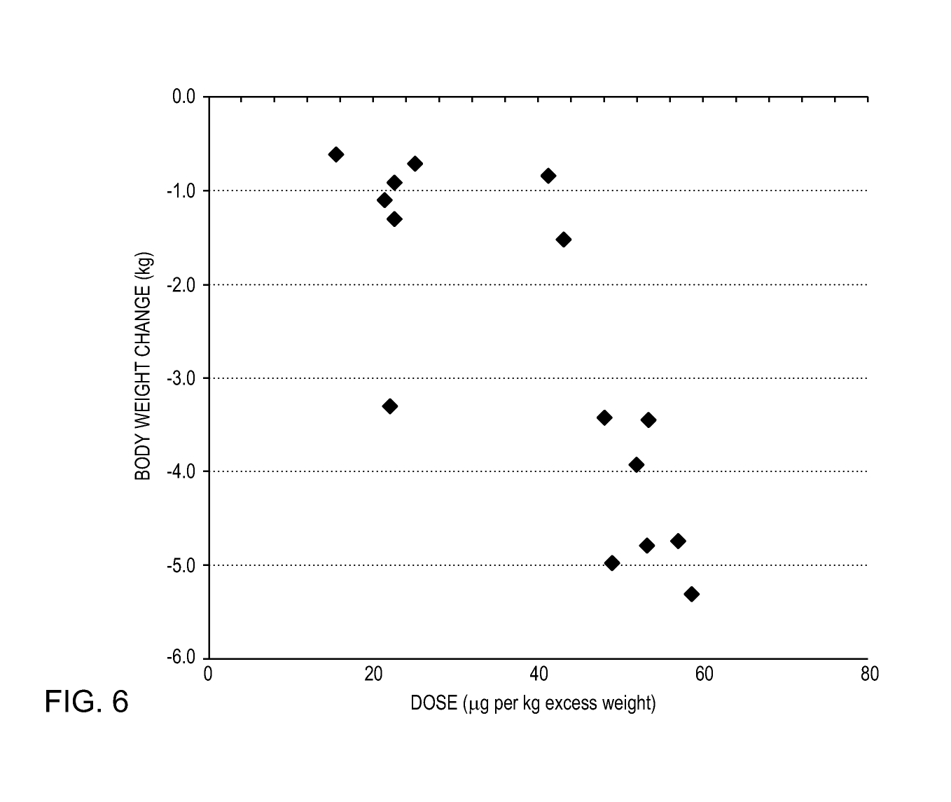

All of the subjects of the trial described in Example 1 received doses ranging from 1.8 to 2.2 mg with each administration (given twice weekly). However, weight change of was not strictly associated with dose delivered. Instead, response was strongly associated with dose administered per unit excess body weight, as compared to weight change associated with dose delivered per unit body weight. FIG. 6 indicates the body weight change of patients in the combined 0.1 and 0.3 mg/m² cohorts vs. dose of compound in μg per kg excess weight of patient, and indicates that exceeding approximately 40 μg of drug per kg of excess body weight may be important in order to see effects in obese patients.

This correlation may be due to obtaining the drug effect at least in part by exposure of the drug to adipose tissue. This indicates that dosing appropriately with a MetAP2 inhibitor surprisingly requires consideration of a patient's excess weight (instead of total weight), given that drug exposures (drug amount) were not substantially different between subjects and both the vasculature and liver were dosed equivalently, but weight loss differed.

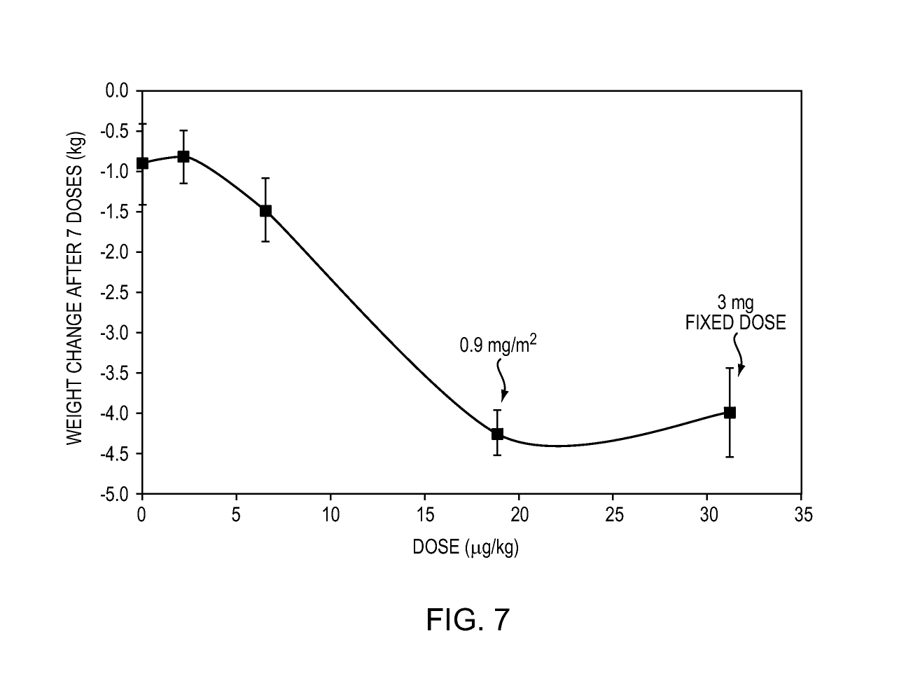

FIG. 7 indicates that weight loss efficacy is obtained at doses of ˜20 μg/kg, and above, administered twice weekly.

### Example 3

### Adiponectin Levels and Effective Dose

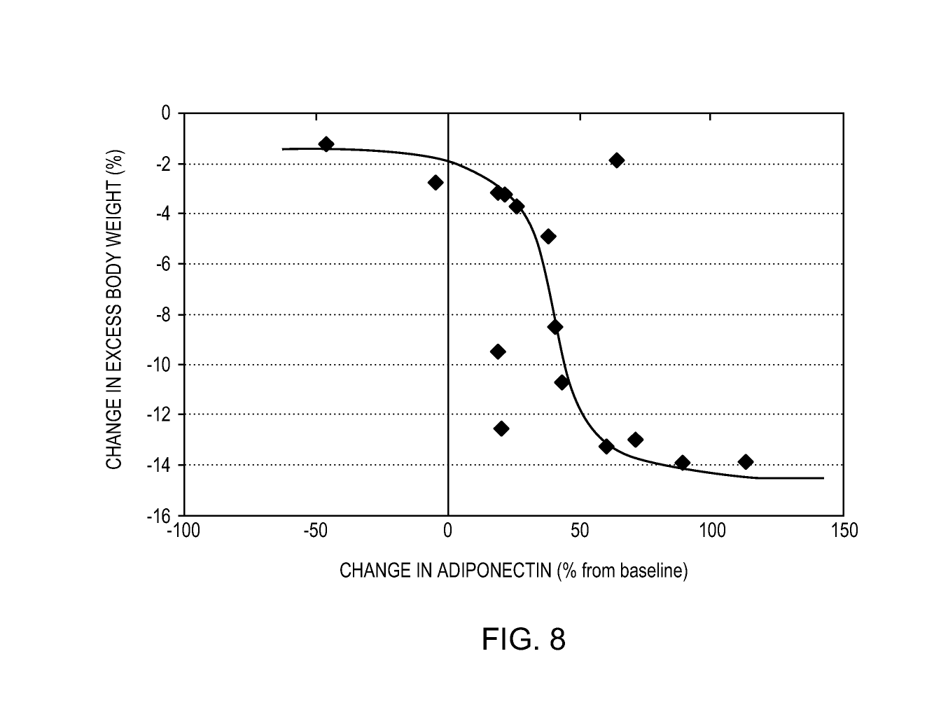

Levels of adiponectin in the serum of patients undergoing the trial were also measured. Adiponectin concentrations were markedly increased (by over 60%, see FIG. 6) with treatment, also pointing to the importance of adipose as a target for the drug since adiponectin is produced by fat cells. FIG. 8 also indicates that there is a strong correlation between adiponectin changes and excess weight loss.

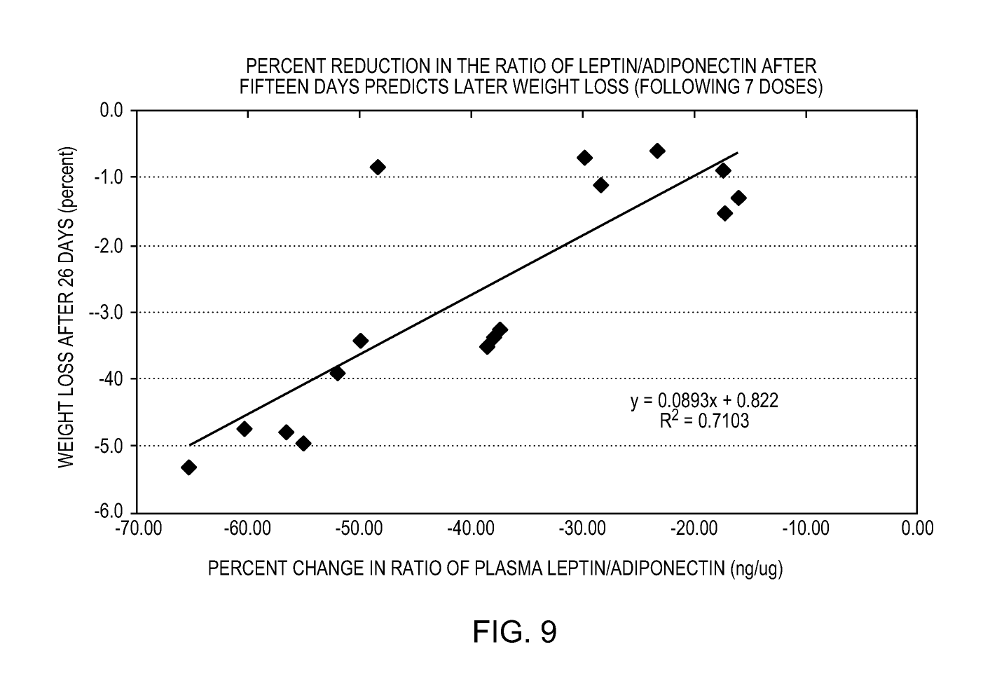

FIG. 9 indicates the changes in the ratio of two adipocyte factors (leptin and adiponectin) for subjects in the 0.3 and 0.9 mg/m² cohorts after 26 days of the trial. The effect of treatment on the ratio of leptin to adiponectin appears to be a particularly strong predictor of weight. Combined, these observations appear to indicate that adipose tissue is a critical target related to weight loss, and that targeting fat tissue for MetAP2 inhibitors or dosing to achieve optimal fat tissue exposure is important.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of treating obesity in a patient in need thereof, comprising administering an effective amount of a MetAP-2 inhibitor, wherein the effective amount is proportional to excess body weight or excess adipose tissue of said patient.

2. A method of treating obesity in a patient in need thereof, comprising:
a) determining the excess body weight or excess adipose tissue of said patient;
b) determining an effective dose of a MetAP-2 based on the excess body weight or adipose tissue for said patient;
c) administering the effective dose to said patient.

3. A method of treating obesity in a patient in need thereof comprising administering to a patient an effective amount of a MetAP-2 inhibitor, wherein the effective amount is capable of increasing adiponectin levels in the patient by at least 50% above the adiponectin level in the patient before administration of the inhibitor.

4. The method of claim 3, wherein the effective amount is capable of increasing adiponectin levels by 60% or more as compared to adiponectin levels in the patient before treatment.

5. The method of claim 3, wherein the effective amount reduces the ratio of leptin to adiponectin plasma levels in the patient after administration.

6. A method of optimizing weight loss in a patient undergoing weight loss treatment, comprising:
a) administering an amount of a MetAP-2 inhibitor to said patient;
b) determining the increase in adiponectin in said patient;
c) increasing the amount of the MetAP-2 inhibitor administered to the patient if the change in adiponectin in the patient is less than an increase of about 60% or more as compared to the adiponectin level of the patient before administration of the MetAP-2 inhibitor.

7. A method of optimizing weight loss in a patient undergoing weight loss treatment, comprising:
a) administering an amount of a MetAP-2 inhibitor to said patient;
b) determining the increase in adiponectin in said patient;
c) increasing the amount of the MetAP-2 inhibitor administered to the patient if a reduction in the ratio of leptin to adiponectin in the plasma of the patient is not greater than 50%.

8. The method of claim 1, wherein said MetAP-2 inhibitor is a substantially irreversible inhibitor.

9. The method of claim 1, wherein said MetAP-2 inhibitor is selected from the group consisting of a fumagillin, fumagillol, fumagillol ester, or fumagillin ketone, siRNA, shRNA, an antibody, or a antisense compound.

10. The method of claim 1, wherein said MetAP-2 inhibitor is selected from O-(4-dimethylaminoethoxycinnamoyl)fumagillol and pharmaceutically acceptable salts thereof.

11. The method of claim 1, wherein the MetAP-2 inhibitor is a reversible inhibitor.

12. The method of claim 1, wherein the patient is human.

13. The method of claim 1, wherein the patient is a companion animal.

14. The method of claim 12, wherein the patient has an initial body mass index of at least about 27 kg/m2.

15. The method of claim 12, wherein the patient has an initial body mass index of at least about 30 kg/m2.

16. The method of claim 12, wherein the patient has a body mass index of at least about 35 kg/m2.

17. The method of claim 1, wherein administering comprises subcutaneous administration.

18. The method of claim 1, wherein administering comprises intravenous administration.
